(19) United States
(12) Patent Application Publication
(10) Pub. No.: US 2008/0027150 A1
(43) Pub. Date: Jan. 31, 2008

(54) Hydrocarbons Synthesis

(76) Inventor: Andre Peter Steynberg, Vanderbijlpark (ZA)

Correspondence Address: LADAS & PARRY, 26 WEST 61ST STREET, NEW YORK, NY, 10023, US

(21) Appl. No.: 10/588,476

(22) PCT Filed: Feb. 3, 2005
(86) PCT No.: PCT/IB05/50448

§ 371 Date: May 9, 2007

Related U.S. Application Data
(60) Provisional application No. 60/542,089, filed on Feb. 5, 2004.

(51) Int. Cl. C07C 27/00

(52) U.S. Cl. 518/700

(57) ABSTRACT

A process for synthesising hydrocarbons includes feeding a gaseous feedstock comprising hydrogen, carbon monoxide and carbon dioxide, into a dimethyl ether (DME) synthesis stage, and in the DME synthesis stage, converting a portion of the gaseous feedstock into a DME product and gaseous products. The DME product is separated from unreacted gaseous reactants and the gaseous products to obtain a tail gas comprising hydrogen and carbon monoxide. The tail gas is fed into a Fischer-Tropsch hydrocarbon synthesis stage, and the hydrogen, carbon monoxide and carbon dioxide are allowed at least partially to react catalytically in the Fischer-Tropsch hydrocarbon synthesis stage to form hydrocarbons.

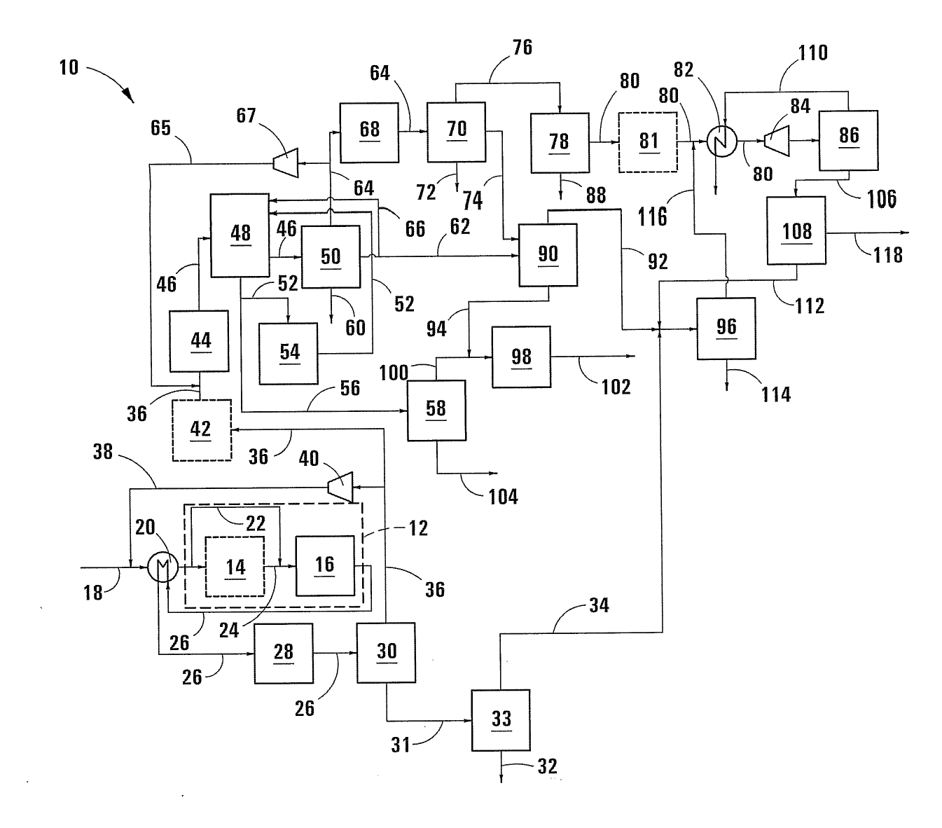

THIS INVENTION relates to hydrocarbon synthesis. In particular, it relates to a process for synthesising hydrocarbons.

According to the invention, there is provided a process for synthesising hydrocarbons, which process includes
feeding a gaseous feedstock comprising hydrogen, carbon monoxide and carbon dioxide, into a dimethyl ether (DME) synthesis stage;
in the DME synthesis stage, converting a portion of the gaseous feedstock into a DME product and gaseous products;
separating the DME product from unreacted gaseous reactants and the gaseous products to obtain a tail gas comprising hydrogen and carbon monoxide;
feeding the tail gas into a Fischer-Tropsch hydrocarbon synthesis stage; and
allowing the hydrogen, carbon monoxide and carbon dioxide at least partially to react catalytically in the Fischer-Tropsch hydrocarbon synthesis stage to form hydrocarbons.

The Fischer-Tropsch hydrocarbon synthesis stage may be a two-phase high temperature catalytic Fischer-Tropsch hydrocarbon synthesis stage, the hydrocarbons formed in the Fischer-Tropsch hydrocarbon synthesis stage thus being gaseous hydrocarbons at the operating pressure and temperature of the Fischer-Tropsch hydrocarbon synthesis stage.

The process may include adjusting the composition of the gaseous feedstock so that the gaseous feedstock has a syngas number (SN) between 1.8 and 2.2, where

$$SN = \frac{[ {H}_{2} ] - [ {CO}_{2} ]}{[ CO ] + [ {CO}_{2} ]}$$

and where [H₂], [CO] and [CO₂] respectively are the molar proportions of hydrogen, carbon monoxide and carbon dioxide in the gaseous feedstock.

Preferably, the syngas number is between 1.85 and 2.15, more preferably between 1.9 and 2.1, e.g. about 2.

Adjusting the composition of the gaseous feedstock may include removing some CO₂ from the gaseous feedstock. Thus, the syngas number may be adjusted upwardly. As will be appreciated, the gaseous feedstock can be derived from a methane-containing gas such as natural gas, or it can be derived from solid carbonaceous material such as coal. When the gaseous feedstock is derived from a carbonaceous material, such as coal, it is expected that, in a preferred embodiment of the process, CO₂ will be removed from the gaseous feedstock. However, CO₂ may also be removed from the gaseous feedstock when the gaseous feedstock is derived from a methane-containing gas.

Removing some CO₂ from the gaseous feedstock may include absorbing CO₂ in an absorbent or solvent, e.g. a Benfield solution. The process may thus also include recovering the removed CO₂, by stripping the CO₂ from the solvent. This may be achieved, for example, by using a stripping gas and elevating the temperature of the solvent. The stripping gas may be used as gaseous feedstock to the Fischer-Tropsch hydrocarbon synthesis stage.

Instead, or in addition, adjusting the composition of the gaseous feedstock may include adding an H₂ rich gas to the gaseous feedstock.

A suitable H₂ rich gas may be obtained by recovering H₂ from a tail gas from the Fischer-Tropsch hydrocarbon synthesis stage. This may be accomplished using pressure swing adsorption (PSA) or cold separation.

A suitable H₂ rich gas may also be obtained by subjecting synthesis gas to the water gas shift reaction CO+H₂O⇄CO₂+H₂ and thereafter removing CO₂ from the shifted gas. A suitable synthesis gas feed to a shift reactor may be provided by the gaseous feedstock to the DME synthesis stage, tail gas from the DME or Fischer-Tropsch synthesis stages or any other suitable source of synthesis gas.

Adding an H₂ rich gas to the gaseous feedstock may include reforming a portion of the gaseous feedstock in a steam reforming stage to produce an H₂ rich reformed gas, and combining at least some of the H₂ rich reformed gas with the gaseous feedstock being fed to the DME synthesis stage.

Typically, an installation for synthesising hydrocarbons which employs a Fischer-Tropsch hydrocarbon synthesis stage includes a hydroprocessing facility, which in turn relies on a steam reforming facility to generate H₂ for hydroprocessing. Advantageously, the process of the invention can thus rely on such a steam reforming facility, possibly upgraded if necessary, also to provide H₂ rich reformed gas with which the composition of the gaseous feedstock can be adjusted, if necessary.

As mentioned hereinbefore, the gaseous feedstock may be derived from a methane-containing gas. Derivation of the gaseous feedstock may include reforming the methane-containing gas in a reforming stage in the presence of oxygen and steam. The reforming stage may be an autothermal reforming stage. Preferably, a low steam to carbon ratio of between about 0.2 and about 0.6, e.g. about 0.4, is used in the autothermal reforming stage. Instead, the reforming stage may be a catalytic or a non-catalytic partial oxidation stage, in which a steam to carbon ratio of 0.2 or less is typically used.

When derived from a methane-containing gas, the gaseous feedstock may comprise hydrogen and carbon monoxide in a molar ratio of between about 1.5 and about 2.3. When derived from a solid carbonaceous material, and relying on gasification of the solid carbonaceous material in a gasification stage, the gaseous feedstock typically has an H₂/CO molar ratio of between about 0.4 and about 2.1, often between about 0.7 and about 2.0.

Converting a portion of the gaseous feedstock into a DME product and gaseous products typically includes contacting the gaseous feedstock with a catalyst or catalysts that enhance or promote methanol synthesis and methanol dehydration reactions. The DME synthesis stage may thus include a methanol reactor followed by a combined methanol synthesis and methanol dehydration reactor.

As methanol catalyst, a copper-containing catalyst is usually employed. Suitable catalysts however include compositions containing copper, zinc oxide, chromia, and/or alumina and possibly other oxidic materials such as magnesia.

Methanol dehydration catalysts usually comprise alumina or alumina silicates as active compounds.

The DME product thus typically includes a mixture of DME and methanol, e.g. with a DME and methanol molar ratio of about 1:1. If desired, the DME product can be subjected to a rectification process to recover a DME product with a desired purity. Typically, however, the process includes converting the DME product into light olefins, e.g. C₂-C₄ olefins, in a light olefins production stage without increasing the DME concentration in the DME product.

The process may include recycling a portion of the tail gas from the DME synthesis stage to the DME synthesis stage. Typically, this recycle is less than the recycle encountered in a conventional stand-alone process for the production of DME. Thus, it is expected that a suitable ratio of tail gas recycle to gaseous feedstock will be between about 0:1 and about 2:1, preferably about 1:1.

The DME synthesis stage may be operated at conditions suitable to ensure that overall CO+CO₂ conversion in the DME synthesis stage is between about 20% and about 80%.

Thus, the DME synthesis stage may be operated at a pressure of between about 50 bar(g) and about 100 bar(g), preferably at a pressure of about 100 bar(g).

The tail gas from the DME synthesis stage typically includes unreacted hydrogen, unreacted carbon monoxide, carbon dioxide, and possibly other gaseous products. Advantageously, the carbon monoxide, carbon dioxide and hydrogen are then converted in the Fischer-Tropsch hydrocarbon synthesis stage to valuable hydrocarbons.

The gaseous hydrocarbons and any unreacted hydrogen, unreacted carbon monoxide, and CO₂ are withdrawn from the Fischer-Tropsch hydrocarbon synthesis stage, and may be separated into one or more condensed liquid hydrocarbon streams, a reaction water stream and a Fischer-Tropsch hydrocarbon synthesis stage tail gas.

The process typically includes recycling some of the Fischer-Tropsch hydrocarbon synthesis stage tail gas to the Fischer-Tropsch hydrocarbon synthesis stage, to obtain high overall CO+CO₂ conversions in the Fischer-Tropsch hydrocarbon synthesis stage. For the Fischer-Tropsch hydrocarbon synthesis stage, overall CO+CO₂ conversion may be at least 80%, preferably at least 85%.

The ratio of the Fischer-Tropsch hydrocarbon synthesis stage tail gas recycle to the tail gas from the DME synthesis stage fed to the Fischer-Tropsch hydrocarbon synthesis stage may be between about 2.5:1 and about 1:1.5, e.g. about 2:1.

The Fischer-Tropsch hydrocarbon synthesis stage may operate at a temperature of at least 320° C. Typically, the Fischer-Tropsch hydrocarbon synthesis stage operates at a temperature between about 320° C. and 350° C., e.g. about 350° C., and at an operating pressure in the range 10 bar to about 50 bar, i.e. at a lower operating pressure than the DME synthesis stage, e.g. 25 bar.

The Fischer-Tropsch hydrocarbon synthesis stage is thus a low chain growth synthesis stage, which typically employs a two-phase fluidised bed reactor and which does not produce a continuous liquid hydrocarbon product phase in the fluidised bed reactor.

The Fischer-Tropsch catalyst used in the Fischer-Tropsch hydrocarbon synthesis stage may be an iron catalyst, and is preferably a promoted iron catalyst. The catalyst may be promoted for activity and/or selectivity.

The DME synthesis stage tail gas fed to the Fischer-Tropsch hydrocarbon synthesis stage may comprise hydrogen, carbon monoxide and carbon dioxide with a syngas number (SN) between about 1.85 and about 2.15, typically between about 1.9 and about 2.1, e.g. about 2.

The process preferably includes, in a separation stage, separating light hydrocarbons, e.g. C₂-C₄ hydrocarbons, from the Fischer-Tropsch hydrocarbon synthesis stage tail gas. These light hydrocarbons may be converted, together with the DME product, into light olefins in the light olefins production stage.

The process may include treating the condensed liquid hydrocarbons from the Fischer-Tropsch hydrocarbon synthesis stage, to provide a light hydrocarbon fraction, including naphtha, which may be converted, together with the DME product, in the light olefin production stage to light olefins, and to provide a diesel fraction.

Separation equipment may be used to recover C₂-C₄ light olefins from the Fischer-Tropsch hydrocarbon synthesis stage. C₂-C₄ light olefins from the light olefins production stage may be recovered using the same separation equipment that is used to recover the C₂-C₄ light olefins produced by Fischer-Tropsch synthesis.

The process may include a diesel hydrotreatment stage to produce high quality diesel motor fuel from one or more diesel factions produced by the process of the invention.

In the light olefin production stage, the DME product and/or the light hydrocarbon fraction from the condensed liquid hydrocarbons produced by the Fischer-Tropsch hydrocarbon synthesis stage and/or the light hydrocarbons from the Fischer-Tropsch hydrocarbon synthesis stage tail gas is converted into light olefins, such as ethylene and propylene. Preferably, a zeolite such as ZSM-5 or a molecular sieve catalyst, preferably a silicoalumina phosphate catalyst is used to produce the light olefins. Suitable silicoalumina phosphate catalysts include SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47 and SAPO-56, the metal-containing forms thereof, and mixtures thereof.

The invention will now be described, by way of example, with reference to the accompanying diagrammatic drawing which shows a simplified flow sheet of a process in accordance with the invention for synthesising hydrocarbons.

Referring to the drawing, reference numeral 10 generally indicates a process in accordance with the invention for synthesising hydrocarbons, such as Fischer-Tropsch derived diesel and light olefins such as ethylene and propylene.

The process 10 includes a DME synthesis stage 12 comprising a methanol reactor 14 and a combined methanol and DME reactor 16. A syngas feed line 18 feeds into a heat exchanger 20 and from the heat exchanger 20 into the methanol reactor 14, with a bypass line 22 being provided around the methanol reactor 14. A methanol feed line 24 connects the methanol reactor 14 and the methanol and DME reactor 16.

A raw DME product line 26 leaves the methanol and DME reactor 16 and passes through the heat exchanger 20 and a cooler 28 before entering a vapour-liquid separator 30. The vapour-liquid separator 30 is provided with a liquid product line 31 and a tail gas line 36. The liquid product line 31 is fed to a fractionation stage 33 provided with a water withdrawal line 32 and a DME product line 34.

A tail gas recycle line 38 branches from the tail gas line 36 and passes through a compressor 40 before returning to the syngas feed line 18.

The tail gas line 36 passes through an optional heater 42 before entering a high temperature Fischer-Tropsch hydrocarbon synthesis stage 44. A gaseous product line 46 leads from the synthesis stage 44 to a washing column 48 and from the washing column 48 to a three-phase separator 50, via a cooler (not shown). A tail gas line 64 leaves the separator 50. A tail gas recycle line 65 branches from the tail gas line 64 and passes through a compressor 67 before returning to the tail gas line 36 entering the hydrocarbon synthesis stage 44.

The washing column 48 is provided with a heavy oil recycle line 52 and a cooler 54. The heavy oil recycle line 52 is taken from a heavy oil line 56 which leads from a bottom of the washing column 48 to a vacuum distillation stage 58.

A reaction water line 60, a hydrocarbon condensate line 62 and the Fischer-Tropsch hydrocarbon synthesis stage tail gas line 64 lead from the separator 50. A hydrocarbon condensate reflux line 66 is taken from the hydrocarbon condensate line 62 and leads back into the washing column 48.

The tail gas line 64 leads into a refrigeration stage 68 and from there into a separator 70. A tail gas aqueous condensate line 72, a tail gas hydrocarbon condensate line 74 and a wet tail gas line 76 leave the separator 70. The wet tail gas line 76 feeds into a dryer 78. From the dryer 78, a dry tail gas line 80 passes through an optional CO₂ removal stage 81, a heat exchanger 82 and an expansion turbine 84 before entering another separator 86. The dryer 78 is also provided with a water withdrawal line 88.

The hydrocarbon condensate line 62 and the tail gas hydrocarbon condensate line 74 lead to an atmospheric distillation stage 90. From the atmospheric distillation stage 90, a light hydrocarbon fraction line 92 and a diesel fraction line 94 respectively lead to a light olefin production stage 96 and a diesel hydrotreatment stage 98. The diesel hydrotreatment stage 98 is also fed with a diesel fraction line 100 from the vacuum distillation stage 58. A diesel product line 102 leaves the diesel hydrotreatment stage 98 and a heavy oil product line 104 leaves the vacuum distillation stage 58.

A light hydrocarbon line 106 leads from the separator 86 into an olefin purification stage 108 and a fuel gas line 110 leads from the separator 86 and passes through the heat exchanger 82. A light olefins product line 118 leads from the olefin purification stage 108.

The light olefin production stage 96 is fed with the light hydrocarbon fraction line 92 from the atmospheric distillation stage 90, the DME product line 34 from the fractionation stage 33 and a paraffins and heavy hydrocarbons line 112 from the olefin purification stage 108. A water withdrawal line 114 and an olefins line 116 lead from the light olefin production stage 96. The olefins line 116 leads from the light olefin production stage 96 into the dry tail gas line 80, before the heat exchanger 82.

In use, syngas with a syngas number between 1.8 and 2.2, e.g. about 2, is fed along the syngas feed line 18 into the methanol reactor 14 with a portion, e.g. 15% fed directly to the methanol and DME reactor 16 along bypass line 22. Before entering the reactor 14 or 16, the syngas is heated in the heat exchanger 20 to a temperature of about 200° C. The syngas, comprising CO, CO₂ and H₂, is typically at a pressure of about 100 bar(g).

The syngas can be derived from natural gas or from solid carbonaceous material. When derived from natural gas, the syngas is typically obtained by subjecting the natural gas to a partial oxidation reforming step or autothermal reforming step operating with a low steam to carbon ratio to produce a synthesis gas with an H₂:CO ratio of less than 2.4. If necessary, the composition of the syngas is adjusted to obtain a syngas number between 1.8 and 2.2, e.g. by the addition of an H₂ rich gas obtained from a steam reformer unit.

In the methanol reactor 14, the syngas is contacted with a copper-containing catalyst to produce methanol. The methanol and unreacted syngas are then fed, by means of the methanol feed line 24 together with the bypassed synthesis gas to the methanol and DME reactor 16, to produce a raw DME product comprising methanol and DME and water. In the methanol and DME reactor 16, the methanol and syngas mixture is contacted with a methanol catalyst and a methanol dehydration catalyst, thereby providing a product mixture with a DME to methanol ratio of approximately 1:1 on a molar basis. The methanol dehydration catalyst is typically a catalyst comprising alumina or alumina silicates as active compounds.

The raw DME product from the methanol and DME reactor 16 leaves the reactor 16 by means of the raw DME product line 26 and exchanges heat in indirect relationship with the syngas in the syngas feed line 18, by means of the heat exchanger 20, before entering the cooler 28, where it is cooled and then fed to the vapour-liquid separator 30.

In the vapour-liquid separator 30, liquid reaction products are separated from gaseous or uncondensed products and unreacted reactants and removed along the line 31 and any uncondensed components are removed as a tail gas along the tail gas line 36. The liquid reaction products are fed to the fractionation stage 33 where water is separated from a DME product comprising DME and methanol. The water is removed along the water withdrawal line 32. The DME product is removed by means of the DME product line 34. A portion of the tail gas in the tail gas line 36 from the separator 30 is recycled by means of the tail gas recycle line 38 and compressor 40 to the syngas feed line 18. Typically, the ratio of tail gas recycle to syngas is about 1.1:1, providing an overall CO+CO₂ conversion in the DME synthesis stage 12 of the order of about 50%.

The tail gas from the separator 30 not recycled is optionally heated in the heater 42 before entering the high temperature Fischer-Tropsch hydrocarbon synthesis stage 44. The tail gas comprises unreacted hydrogen, unreacted carbon monoxide and carbon dioxide and requires no composition adjustment before entering the hydrocarbon synthesis stage 44. The tail gas may also contain uncondensed DME. Preferably, the Fischer-Tropsch hydrocarbon synthesis stage 44 is operated at a lower pressure that the DME synthesis stage 12, so that no additional compression of the tail gas fed to the synthesis stage 44 is required.

The high temperature Fischer-Tropsch hydrocarbon synthesis stage 44 typically comprises one or more two-phase fluidised bed reactors operating at a high Fischer-Tropsch hydrocarbon synthesis reaction temperature typically between about 320° C. and 350° C. In these fluidised bed reactors, the carbon monoxide, carbon dioxide and hydrogen react to form gaseous hydrocarbons which leave the hydrocarbon synthesis stage 44 along the gaseous product line 46. The catalyst used in the hydrocarbon synthesis stage 44 is a promoted iron catalyst. The operation of a high temperature Fischer-Tropsch synthesis stage, such as the hydrocarbon synthesis stage 44, is well-known to those skilled in the art and will not be described in further detail.

The gaseous hydrocarbons from the hydrocarbon synthesis stage 44 enter the washing column 48 which uses heavy oil, and hydrocarbon concentrate from the separator 50, as a washing liquid. The heavy oil is circulated through the cooler 54 which removes heat introduced by the gaseous hydrocarbons from the hydrocarbon synthesis stage 44.

Gaseous hydrocarbons passing through the washing column 48 leave the washing column 48 by means of the gaseous product line 46 and are cooled in a cooler (not shown) before entering the separator 50. Before entering the separator 50, the gaseous hydrocarbons are thus cooled to a temperature of between about 30° C. and about 80° C., e.g. about 70° C. In the cooler and the separator 50, reaction water condenses and, after separation, is removed along the reaction water line 60. Some hydrocarbons also condense to form a hydrocarbon condensate, which is removed along the hydrocarbon condensate line 62. The remaining gaseous hydrocarbons leave the separator 50 as a tail gas, along the tail gas line 64.

In the Fischer-Tropsch hydrocarbon synthesis stage 44, preferably up to about 85% of the CO and CO₂ entering the stage 44 is converted to hydrocarbons. In order to achieve such high conversion rates, a portion of the tail gas in the tail gas line 64 from the separator 50 is recycled, by means of the tail gas recycle line 65 and the compressor 67. Typically, the ratio of tail gas from the Fischer-Tropsch hydrocarbon stage 44 to tail gas from the DME synthesis stage 12, fed to the Fischer-Tropsch hydrocarbon synthesis stage 44, is about 2:1.

The tail gas from the separator 50 which is not recycled to the hydrocarbon synthesis stage 44, is refrigerated in the refrigeration stage 68 typically to a temperature of about 5° C. The refrigerated tail gas then enters the separator 70, by means of the tail gas line 64. In the separator 70, the refrigerated tail gas is separated into an aqueous tail gas condensate removed along the tail gas aqueous condensate line 72, a tail gas hydrocarbon condensate removed along the tail gas hydrocarbon condensate line 74, and wet tail gas which is removed along the wet tail gas line 76.

The wet tail gas is dried in the dryer 78 and fed by means of the dry tail gas line 80 to the heat exchanger 82 where it is cooled further before passing through the expansion turbine 84 (other expansion or cooling techniques may instead be used), which causes the temperature of the dry tail gas to drop to about −80° C. If desired, the dry tail gas may first pass through the optional CO₂ removal stage 81 to remove and recover CO₂ from the tail gas, using conventional methods known to those skilled in the art.

The cold dry tail gas from the expansion turbine 84 is fed into the separator 86, where it is separated into light liquid hydrocarbons, predominantly comprising light olefins and paraffins, which are removed along the light hydrocarbon line 106, and a hydrocarbon lean tail gas which is removed along the fuel gas line 110 and which passes through the heat exchanger 82 in indirect heat exchange relationship with the dry tail gas in the dry tail gas line 80. Other more complex heat exchange relationships may also be applied.

The light hydrocarbons in the light hydrocarbon line 106 are further separated by separation methods known to those skilled in the art, in the olefin purification stage 108, to provide a light olefins product which is withdrawn along the light olefins product line 118. Typically, the light olefins product includes ethylene, propylene and butylene. Paraffins, such as C₂-C₄ paraffins and heavier hydrocarbons are removed from the olefin purification stage 108 by means of the paraffins and heavy hydrocarbon line 112.

The hydrocarbon condensate from the three-phase separator 50 and the tail gas hydrocarbon condensate from the separator 70 are fed by means of the lines 62, 74 to the atmospheric distillation stage 90 where the hydrocarbon condensate is distilled into various fractions, as desired. In contrast, the heavy oil from the washing column 48 is fed by means of the heavy oil line 56 to the vacuum distillation stage 58 where it is distilled under a vacuum into various desired fractions.

The vacuum distillation stage 58 produces a heavy oil product which is removed along the heavy oil product line 104 for further processing and/or purification, and a diesel fraction which is removed along the diesel fraction line 100. The atmospheric distillation stage 90 produces a light hydrocarbon fraction, comprising naphtha and other light hydrocarbons, which is removed along the light hydrocarbon fraction line 92, and a diesel fraction which is removed along the diesel fraction line 94. The diesel fractions from the vacuum distillation stage 58 and the atmospheric distillation stage 90 are fed to the diesel hydrotreatment stage 98, which is supplied with hydrogen (not shown), to provide a diesel product which is withdrawn along the diesel product line 102.

The DME product in the DME product line 34, the light hydrocarbon fraction in the light hydrocarbon fraction line 92, and the paraffins and heavy hydrocarbons in the paraffins and heavy hydrocarbons line 112 are fed to the light olefin production stage 96 as a feedstock. The feedstock is passed over a DME dehydration catalyst, such as ZSM-5 or SAPO-34. In the process, the feedstock is dehydrated, producing an aqueous condensate stream which is removed along the water withdrawal line 114, and light olefins which are removed along the olefins line 116. The light olefins typically include ethylene, propylene and possibly butylene and small amounts of aromatics and are fed by means of the olefins line 116, via the heat exchanger 82, expansion turbine 84 and separator 86 to the olefin purification stage 108 for purification before withdrawal along the light olefins product line 118.

Advantageously, a favourable pressure gradient exists between the DME synthesis stage 12 and the high temperature Fischer-Tropsch hydrocarbon synthesis stage 44. Also advantageously, as a result of the presence of the high temperature Fischer-Tropsch hydrocarbon synthesis stage 44, the DME synthesis stage tail gas recycle can be reduced, meaning that the capacity of the compressor 40 can be decreased. In other words, the fact that the DME synthesis stage 12 is followed by the high temperature Fischer-Tropsch hydrocarbon synthesis stage 44 means that the syngas conversion required in the DME synthesis stage 12 can be lower than that required for a conventional standalone DME plant, as the unconverted syngas can be converted to a high degree to valuable hydrocarbons in the Fischer-Tropsch hydrocarbon synthesis stage 44 which is non-equilibrium limited. It is also an advantage of the process 10, as illustrated, that no adjustment of the tail gas from the DME synthesis stage 12 is required before the tail gas can be fed to the high temperature Fischer-Tropsch hydrocarbon synthesis stage 44, and that any CO₂ formed in the DME synthesis stage 12 is reactive in the Fischer-Tropsch hydrocarbon synthesis stage 44. The process 10, as illustrated, furthermore allows co-processing of DME with high temperature Fischer-Tropsch naphtha to produce C₂-C₄ olefins. In the process 10, as illustrated, the yield to light olefins is thus higher than for standalone high temperature Fischer-Tropsch hydrocarbon synthesis plants, and at the same time the capital cost related to DME production is lower than that of a standalone DME plant.

### EXAMPLE 1

A stand-alone DME process was modelled using a computerised simulation to set a base case for comparison with the improvement derived from the present invention.

The simulated DME process consists of a cooled methanol reactor followed by an adiabatic combined methanol synthesis and dehydration reactor that contains a bed of dual function catalyst (i.e. combined methanol formation and methanol dehydration) and a bed of methanol dehydration catalyst. The process operates at a pressure of 100 bar. The molar composition of the fresh synthesis gas is 66.2% hydrogen, 24.7% carbon monoxide, 5.2% carbon dioxide and 0.2% water. This corresponds to a syngas number of 2.05.

Recycled synthesis gas is mixed with fresh synthesis gas and preheated to 225° C. 15% of the preheated stream is split from the preheated stream (forming a bypass stream) prior to feeding the remaining 85% to the methanol reactor. The outlet temperature from the methanol reactor is controlled to 274° C. The effluent from the methanol reactor is mixed with the bypass stream and fed to the combined synthesis and dehydration reactor. The effluent from the combined synthesis and dehydration reactor is cooled to condense approximately 99% of the water and methanol and 20% of the DME. The uncondensed gas is split into a recycle stream (93%) and a purge stream (7%). The recycle stream is admixed with the fresh synthesis gas. The purge stream is subjected to an additional cooling step to remove all of the DME.

With a recycle ratio of 2.9 and a per pass H₂ and CO conversion of 27.5%, an overall H₂ and CO conversion of 84.4% and an overall CO and CO₂ conversion of 87.7% is achieved. The mass ratio of methanol product to DME product achieved is 1:1.56. The actual yield over maximum possible yield is 84%.

### EXAMPLE 2

In a comparative example to illustrate the benefits of the present invention, a process in which a natural gas-based feed is partially converted to DME and the tail gas converted to hydrocarbons in a two-phase high temperature Fischer-Tropsch reaction stage, was modelled using a computerised simulation.

A typical synthesis gas composition ex an autothermal reformer was used as fresh synthesis gas, i.e. a molar composition of 64.3% hydrogen, 28.6% carbon monoxide, 3.3% carbon dioxide, 2.3% methane and 1.5% inerts. A hydrogen rich gas with a molar composition of 55.3% hydrogen, 2.1% carbon monoxide, 29.9% methane, 12.4% inerts and 0.3% heavier hydrocarbons is separated from a Fischer-Tropsch synthesis stage tail gas (see below). This hydrogen-rich gas is mixed with the fresh feed gas to yield a feedstock to the DME reaction stage with a syngas number of 2.03. The operation of the DME synthesis stage is similar to that described in example 1, except that a lower overall conversion of reactants is targeted. The DME synthesis stage is operated with a recycle ratio of 1.1 and a per pass H₂ and CO conversion of 28%. In this manner an overall conversion over the DME synthesis stage of 50.2% and 50.7% is achieved for H₂ and CO, and CO and CO₂ respectively.

The tail gas from the DME synthesis stage now serves as feedstock for the Fischer-Tropsch synthesis stage, without the need for any composition adjustment. The DME that may still be present in the tail gas from the DME synthesis stage is passed through to the Fischer-Tropsch synthesis stage. The Fischer-Tropsch synthesis stage includes a Fischer-Tropsch reactor which operates at a pressure of 25 bar and a temperature of 350° C. Tail gas from the Fischer-Tropsch reactor is treated to recover hydrocarbons and water. The Fischer-Tropsch tail gas is subjected to a first condensation stage at 30 to 70° C., whereafter a potion of the tail gas is recycled to the inlet of the Fischer-Tropsch reactor, while the remainder is subjected to CO₂ removal followed by further cooling and separation in a cold separation unit to recover light C₂+ hydrocarbons. The DME present in the effluent from the Fischer-Tropsch reactor is recovered together with the products from the Fischer-Tropsch synthesis stage. A hydrogen-rich gas is separated in a cold separation unit and used to adjust the syngas number of the fresh synthesis gas to 2.03.

The Fischer-Tropsch synthesis stage is operated with per pass H₂ and CO conversion of 45.6% and a recycle ratio of 2. This results in an overall conversion of 85.7% and 84.7% for H₂ and CO, and CO and CO₂ respectively over the Fischer-Tropsch synthesis stage.

For the process as a whole, the overall H₂ and CO conversion is 96.7%, while the CO and CO₂ conversion is 92.5%.

The mass ratio of products for methanol:DME:hydrocarbons is 1:2.14:0.63.

The actual yield of the process to the maximum theoretical yield is 91%.

1. A process for synthesising hydrocarbons, which process includes
feeding a gaseous feedstock comprising hydrogen, carbon monoxide and carbon dioxide, into a dimethyl ether (DME) synthesis stage;
in the DME synthesis stage, converting a portion of the gaseous feedstock into a DME product and gaseous products;
separating the DME product from unreacted gaseous reactants and the gaseous products to obtain a tail gas comprising hydrogen and carbon monoxide;
feeding the tail gas into a Fischer-Tropsch hydrocarbon synthesis stage; and
allowing the hydrogen, carbon monoxide and carbon dioxide at least partially to react catalytically in the Fischer-Tropsch hydrocarbon synthesis stage to form hydrocarbons.

2. The process as claimed in claim 1, in which the Fischer-Tropsch hydrocarbon synthesis stage is a two-phase high temperature catalytic Fischer-Tropsch hydrocarbon synthesis stage, the hydrocarbons formed in the Fischer-Tropsch hydrocarbon synthesis stage thus being gaseous hydrocarbons at the operating pressure and temperature of the Fischer-Tropsch hydrocarbon synthesis stage.

3. The process as claimed in claim 1 or claim 2, which includes adjusting the composition of the gaseous feedstock so that the gaseous feedstock has a syngas number (SN) between 1.8 and 2.2, where
$$SN = \frac{[ {H}_{2} ] - [ {CO}_{2} ]}{[ CO ] + [ {CO}_{2} ]}$$
and where [H₂], [CO] and [CO₂] respectively are the molar proportions of hydrogen, carbon monoxide and carbon dioxide in the gaseous feedstock.

4. The process as claimed in any one of the preceding claims, in which converting a portion of the gaseous feedstock into a DME product and gaseous products includes contacting the gaseous feedstock with a catalyst or catalysts that enhance methanol synthesis and methanol dehydration reactions.

5. The process as claimed in any one of the preceding claims, in which the DME product includes a mixture of DME and methanol and which includes converting the DME product into light olefins in a light olefins production stage without increasing the DME concentration in the DME product.

6. The process as claimed in any one of the preceding claims, which includes recycling a portion of the tail gas from the DME synthesis stage to the DME synthesis stage, a ratio of tail gas recycle to gaseous feedstock being between about 0:1 and about 2:1.

7. The process as claimed in any one of the preceding claims, in which the DME synthesis stage is operated at conditions suitable to ensure that overall CO+CO₂ conversion in the DME synthesis stage is between about 20% and about 80%.

8. The process as claimed in any one of the preceding claims, which includes recycling some of the Fischer-Tropsch hydrocarbon synthesis stage tail gas to the Fischer-Tropsch hydrocarbon synthesis stage, to obtain high overall CO+CO₂ conversions in the Fischer-Tropsch hydrocarbon synthesis stage of at least 80%.

9. The process as claimed in any one of the preceding claims, which includes recycling some of the Fischer-Tropsch hydrocarbon synthesis stage tail gas to the Fischer-Tropsch hydrocarbon synthesis stage, a ratio of Fischer-Tropsch tail gas recycle to the tail gas from the DME synthesis stage fed to the Fischer-Tropsch hydrocarbon synthesis stage being between 2.5:1 and 1:1.5.

10. The process as claimed in claim 5, which includes, in a separation stage, separating light hydrocarbons from the Fischer-Tropsch hydrocarbon synthesis stage tail gas and converting these light hydrocarbons, together with the DME product, into light olefins with a carbon number from 2 to 4 in the light olefins production stage.

11. The process as claimed in claim 5 or claim 10, in which gaseous hydrocarbons and any unreacted hydrogen, unreacted carbon monoxide, and CO₂ are withdrawn from the Fischer-Tropsch hydrocarbon synthesis stage, and separated into one or more condensed liquid hydrocarbon streams, a reaction water stream and a Fischer-Tropsch hydrocarbon synthesis stage tail gas, the process further including treating the condensed liquid hydrocarbons from the Fischer-Tropsch hydrocarbon synthesis stage, to provide a light hydrocarbon fraction, including naphtha, which is converted, together with the DME product, in the light olefin production stage to light olefins, and to provide a diesel fraction.

12. A process as claimed in claim 5 or claim 10 or claim 11, which includes using separation equipment to recover C₂-C₄ light olefins from the Fischer-Tropsch hydrocarbon synthesis stage and in which C₂-C₄ light olefins from the light olefins production stage are recovered using the same separation equipment that is used to recover the C₂-C₄ light olefins produced by Fischer-Tropsch synthesis.
